SEQUENCE LISTING (64 sequences)

SEQ ID NO: 1 - protein, 29 aa, Adenovirus serotype 5
  DOMAIN (1)..(29): HVR1
EAATALEINLEEEDDDNEDEVDEQAEQQK

SEQ ID NO: 2 - protein, 6 aa, Adenovirus serotype 5
  DOMAIN (1)..(6): HVR2
VEGQTP

SEQ ID NO: 3 - protein, 3 aa, Adenovirus serotype 5
  DOMAIN (1)..(3): HVR3
ETE

SEQ ID NO: 4 - protein, 7 aa, Adenovirus serotype 5
  DOMAIN (1)..(7): HVR4
KQQNGKL

SEQ ID NO: 5 - protein, 13 aa, Adenovirus serotype 5
  DOMAIN (1)..(13): HVR5
TTEATAGNGDNLT

SEQ ID NO: 6 - protein, 3 aa, Adenovirus serotype 5
  DOMAIN (1)..(3): HVR6
IKE

SEQ ID NO: 7 - protein, 14 aa, Adenovirus serotype 5
  DOMAIN (1)..(14): HVR7
KTGQENGWEKDATE

SEQ ID NO: 8 - protein, 182 aa, Adenovirus serotype 5
  DOMAIN (1)..(182): Fiber knob domain
TLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGDTTPSAYSMSFSWDWSGHNYINEIFATSSYTFSYIAQE

SEQ ID NO: 9 - protein, 21 aa, Adenovirus serotype 11
  DOMAIN (1)..(21): HVR1
AEGVKNTTGEEHVTEEETNTT

SEQ ID NO: 10 - protein, 17 aa, Adenovirus serotype 26
  DOMAIN (1)..(17): HVR1
TKEKQGTTGGVQQEKDV

SEQ ID NO: 11 - protein, 24 aa, Adenovirus serotype 34
  DOMAIN (1)..(24): HVR1
DKGVTSTGLVDDGNTDDGEEAKKA

SEQ ID NO: 12 - protein, 24 aa, Adenovirus serotype 35
  DOMAIN (1)..(24): HVR1
AKGVPTAAAAGNGEEEHETEEKTA

SEQ ID NO: 13 - protein, 14 aa, Adenovirus serotype 48
  DOMAIN (1)..(14): HVR1
EKKNGGGSDANQMQ

SEQ ID NO: 14 - protein, 11 aa, Adenovirus serotype 49
  DOMAIN (1)..(11): HVR1
AKENNGQGEAK

SEQ ID NO: 15 - protein, 14 aa, Adenovirus serotype 50
  DOMAIN (1)..(14): HVR1
NKGDEEDGEDDQQA

SEQ ID NO: 16 - protein, 11 aa, Simian adenovirus serotype C68/Pan9
  DOMAIN (1)..(11): HVR1
YKADGETATEK

SEQ ID NO: 17 - protein, 10 aa, Adenovirus serotype 11
  DOMAIN (1)..(10): HVR2
LEVSDEESKP

SEQ ID NO: 18 - protein, 11 aa, Adenovirus serotype 26
  DOMAIN (1)..(11): HVR2
TDETAENGKKD

SEQ ID NO: 19 - protein, 10 aa, Adenovirus serotype 34
  DOMAIN (1)..(10): HVR2
LEVSTEGPKP

SEQ ID NO: 20 - protein, 11 aa, Adenovirus serotype 35
  DOMAIN (1)..(11): HVR2
LEISAENESKP

SEQ ID NO: 21 - protein, 12 aa, Adenovirus serotype 48
  DOMAIN (1)..(12): HVR2
IDATKEEDNGKE

SEQ ID NO: 22 - protein, 13 aa, Adenovirus serotype 49
  DOMAIN (1)..(13): HVR2
IDENKEEDEEGRE

SEQ ID NO: 23 - protein, 11 aa, Adenovirus serotype 50
  DOMAIN (1)..(11): HVR2
LEVPSEGGPKP

SEQ ID NO: 24 - protein, 7 aa, Simian adenovirus serotype C68/Pan9
  DOMAIN (1)..(7): HVR2
TDTDDQP

SEQ ID NO: 25 - protein, 3 aa, Adenovirus serotypes 11/34/35
  DOMAIN (1)..(3): HVR3
DLD

SEQ ID NO: 26 - protein, 3 aa, Adenovirus serotype 26
  DOMAIN (1)..(3): HVR3
ENE

SEQ ID NO: 27 - protein, 3 aa, Adenovirus serotype 48
  DOMAIN (1)..(3): HVR3
DSD

SEQ ID NO: 28 - protein, 3 aa, Adenovirus serotype 49
  DOMAIN (1)..(3): HVR3
NTE

SEQ ID NO: 29 - protein, 3 aa, Adenovirus serotype 50
  DOMAIN (1)..(3): HVR3
DTD

SEQ ID NO: 30 - protein, 3 aa, Simian adenovirus serotype C68/Pan9
  DOMAIN (1)..(3): HVR3
DIT

SEQ ID NO: 31 - protein, 10 aa, Adenovirus serotype 11
  DOMAIN (1)..(10): HVR4
KTTEQPNQKV

SEQ ID NO: 32 - protein, 10 aa, Adenovirus serotype 26
  DOMAIN (1)..(10): HVR4
KPVNEGEQPK

SEQ ID NO: 33 - protein, 11 aa, Adenovirus serotype 34
  DOMAIN (1)..(11): HVR4
KPKEDDGTNNI

SEQ ID NO: 34 - protein, 10 aa, Adenovirus serotype 35
  DOMAIN (1)..(10): HVR4
KNSEPSSEKI

SEQ ID NO: 35 - protein, 11 aa, Adenovirus serotype 48
  DOMAIN (1)..(11): HVR4
KTPEKEGEEPK

SEQ ID NO: 36 - protein, 10 aa, Adenovirus serotype 49
  DOMAIN (1)..(10): HVR4
KTGENGKPTE

SEQ ID NO: 37 - protein, 8 aa, Adenovirus serotype 50
  DOMAIN (1)..(8): HVR4
KKEEEGKV

SEQ ID NO: 38 - protein, 8 aa, Simian adenovirus serotype C68/Pan9
  DOMAIN (1)..(8): HVR4
KTGTGTTK

SEQ ID NO: 39 - protein, 9 aa, Adenovirus serotype 11
  DOMAIN (1)..(9): HVR5
AASQKTNLS

SEQ ID NO: 40 - protein, 16 aa, Adenovirus serotype 26
  DOMAIN (1)..(16): HVR5
VPGGSPPAGGSGEEYK

SEQ ID NO: 41 - protein, 9 aa, Adenovirus serotype 34
  DOMAIN (1)..(9): HVR5
LRSQRSELK

SEQ ID NO: 42 - protein, 9 aa, Adenovirus serotype 35
  DOMAIN (1)..(9): HVR5
NSSQRTNFS

SEQ ID NO: 43 - protein, 16 aa, Adenovirus serotype 48
  DOMAIN (1)..(16): HVR5
IPSTGTGGNGTNVNFK

SEQ ID NO: 44 - protein, 12 aa, Adenovirus serotype 49
  DOMAIN (1)..(12): HVR5
LRQNDTGGNNNQ

SEQ ID NO: 45 - protein, 9 aa, Adenovirus serotype 50
  DOMAIN (1)..(9): HVR5
LRSQMTGLK

SEQ ID NO: 46 - protein, 10 aa, Simian adenovirus serotype C68/Pan9
  DOMAIN (1)..(10): HVR5
NRSAAAAGLA

SEQ ID NO: 47 - protein, 3 aa, Adenovirus serotype 11/35
  DOMAIN (1)..(3): HVR6
TED

SEQ ID NO: 48 - protein, 3 aa, Adenovirus serotype 26/49
  DOMAIN (1)..(3): HVR6
TSD

SEQ ID NO: 49 - protein, 3 aa, Adenovirus serotype 34
  DOMAIN (1)..(3): HVR6
VSD

SEQ ID NO: 50 - protein, 3 aa, Adenovirus serotype 48
  DOMAIN (1)..(3): HVR6
KED

SEQ ID NO: 51 - protein, 3 aa, Adenovirus serotype 50
  DOMAIN (1)..(3): HVR6
ASD

SEQ ID NO: 52 - protein, 3 aa, Simian adenovirus serotype C68/Pan9
  DOMAIN (1)..(3): HVR6
TDD

SEQ ID NO: 53 - protein, 12 aa, Adenovirus serotype 11
  DOMAIN (1)..(12): HVR7
NGDNAPNWKEPE

SEQ ID NO: 54 - protein, 17 aa, Adenovirus serotype 26
  DOMAIN (1)..(17): HVR7
TNGNDGAEESEWEKDDA

SEQ ID NO: 55 - protein, 11 aa, Adenovirus serotype 34
  DOMAIN (1)..(11): HVR7
NGDQSTWTNVD

SEQ ID NO: 56 - protein, 12 aa, Adenovirus serotype 35
  DOMAIN (1)..(12): HVR7
NGEDNNNWKEPE

SEQ ID NO: 57 - protein, 13 aa, Adenovirus serotype 48
  DOMAIN (1)..(13): HVR7
KTTNNTEWEKDTA

SEQ ID NO: 58 - protein, 16 aa, Adenovirus serotype 49
  DOMAIN (1)..(16): HVR7
DTTVAGTNDKWKVNAK

SEQ ID NO: 59 - protein, 12 aa, Adenovirus serotype 50
  DOMAIN (1)..(12): HVR7
NGDETTTWKDLE

SEQ ID NO: 60 - protein, 13 aa, Simian adenovirus serotype C68/Pan9
  DOMAIN (1)..(13): HVR7
NGTDQTTWTKDDS

SEQ ID NO: 61 - protein, 179 aa, Simian adenovirus serotype C68/Pan9
  DOMAIN (1)..(179): Fiber knob domain
TLWTTPDPSPNCQILAENDAKLTLCLTKCGSQILATVSVLVVGSGNLNPITGTVSSAQVFLRFDANGVLLTEHSTLKKYWGYRQGDSIDGTPYTNAVGFMPNLKAYPKSQSSTTKNNIVGQVYMNGDVSKPMLLTITLNGTDDSNSTYSMSFSYTWTNGSYVGATFGANSYTFSYIAQE

SEQ ID NO: 62 - protein, 192 aa, Adenovirus serotype 15
  DOMAIN (1)..(192): Fiber knob domain
TLWTTPDPSPNCRVSEDKDSKLTLILTKCGSQILASFSLLVVKGTYATVDKNTTNKQFSIKLLFDANGKLKSESNLSGYWNYRSDNSVVSTPYDNAVPFMPNTTAYPKIINSTTVPENKKSSAKKTIVGNVYLEGNAGQPVAVAISFNKETTADYSITFDFAWSKAYETPVPFDTSSMTFSYIAQENQDKGE

SEQ ID NO: 63 - protein, 947 aa, Artificial Sequence
  Synthetic construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWEEKKNGGGSDANQMQTHVFGQAPYSGINITKEGIQIGIDATKEEDNGKEIYADKTFQPEPQIGESQWQDSDNYYGGRVLKKTTPMKPCYGSYAKPTNENGGQAKFKTPEKEGEEPKESQVEMQFFDIPSTGTGGNGTNVNFKPKVVLYSEDVDIETPDTHISYMPGKEDASSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLDGAGTNAVYQGVKVKTTNNTEWEKDTAVSEHNQIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHRPHRGVIETVYLRTPFSAGNATT

SEQ ID NO: 64 - protein, 578 aa, Artificial Sequence
  Synthetic construct
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPDPSPNCQILAENDAKLTLCLTKCGSQILATVSVLVVGSGNLNPITGTVSSAQVFLRFDANGVLLTEHSTLKKYWGYRQGDSIDGTPYTNAVGFMPNLKAYPKSQSSTTKNNIVGQVYMNGDVSKPMLLTITLNGTDDSNSTYSMSFSYTWTNGSYVGATFGANSYTFSYIAQE

(19) United States
(12) Patent Application Publication
(10) Pub. No.: US 2014/0348791 A1
(43) Pub. Date: Nov. 27, 2014

(54) MODIFIED ADENOVIRAL VECTORS AND METHODS OF TREATMENT USING SAME

(71) Applicants: Dan H. Barouch, Newton, MA (US); Ritu Bradley, West Newton, MA (US)

(72) Inventors: Dan H. Barouch, Newton, MA (US); Ritu Bradley, West Newton, MA (US)

(73) Assignee: Beth Israel Deaconess Medical Center, Inc., Boston, MA (US)

(21) Appl. No.: 14/343,708

(22) PCT Filed: Sep. 7, 2012
(86) PCT No.: PCT/US12/54201

§ 371 Date: Jun. 27, 2014

Related U.S. Application Data
(60) Provisional application No. 61/533,029, filed on Sep. 9, 2011.

(51) Int. Cl. C12N 15/86

(52) U.S. Cl. CPC C12N 15/86; C12N 2710/10041
USPC 424/93.2; 435/235.1

(57) ABSTRACT

The present invention relates to recombinant adenovirus serotype 5 (Ad5) vectors which harbor chimeric capsid proteins including substitutions of the corresponding regions from adenovirus serotypes having a lower seroprevalence relative to Ad5. In particular, the chimeric capsid includes modifications of both the adenoviral hexon and fiber proteins. The invention also provides methods for the treatment of diseases or disorders caused by infective agent(s) by administering the adenoviral vector(s) to a subject (e.g., a mammal, such as a human).

### BACKGROUND OF THE INVENTION

Recombinant adenoviral vectors have been developed for vaccines. To date, approximately 55 different adenovirus serotypes have been identified. The subgroup C adenoviruses have been most extensively studied for applications such as vaccination and gene therapy. Adenovirus serotypes 2 and 5 (Ad2 and Ad5), in particular, are widely used in the art. Importantly, Ad5 vector-based vaccines have been shown to elicit potent and protective immune responses in a variety of animal models. Moreover, large-scale clinical trials for HIV vaccination using Ad5-based recombinant vectors are ongoing (WO 01/02607; WO 02/22080; Shiver et al., Nature, 2002; Letvin et al., Annu. Rev. Immunol. 20: 73-99, 2002; Shiver and Emini, Annu. Rev. Med. 55: 355, 2004).

The utility of recombinant Ad5 vector-based vaccines for HIV and other pathogens, however, may be limited due to high pre-existing anti-Ad5 immunity in human populations. The existence of anti-Ad5 immunity has been shown to suppress substantially the immunogenicity of Ad5-based vaccines in studies in mice and rhesus monkeys. Early data from phase-1 clinical trials show that this problem may also occur in humans (Shiver, Keystone, 2004). Although both Ad5-specific neutralizing antibodies (NAbs) and CD8+ T lymphocytes contribute to anti-Ad5 immunity, the Ad5-specific NAbs appear to play the primary role in this process (Sumida et al., J. Virol., 2004).

It is therefore important to understand Ad5 immunity and to develop adenoviral vectors that circumvent pre-existing Ad5 immunity. There is an unmet need in the field for alternative adenoviral vectors that evade pre-existing immunities to the adenoviral vector in the host, but that are still immunogenic and capable of inducing strong immune responses against proteins encoded by heterologous nucleic acids carried by the vector.

### SUMMARY OF THE INVENTION

Newly developed recombinant adenoviral vectors for improved gene delivery, prophylactic or therapeutic vaccination, and gene therapy are disclosed herein. In a first aspect, the invention features recombinant, replication-deficient Ad5 vectors having both chimeric hexon and fiber proteins, in which regions of the Ad5 hexon and fiber proteins have been replaced with corresponding regions from adenoviruses having lower seroprevalence compared to that of Ad5. Adenoviruses that are rarely targeted by neutralizing antibodies compared to Ad5, and thus that have lower seroprevalence compared to Ad5, typically include subgroup B (Ad11, Ad34, Ad35, and Ad50) and subgroup D (Ad15, Ad24, Ad26, Ad48, and Ad49) adenoviruses as well as simian adenoviruses (e.g., Pan9, also known as AdC68). In one embodiment, the hexon and fiber proteins of the recombinant, replication-defective Ad5 vector of the invention have been replaced with the corresponding regions from Ad11, Ad15, Ad24, Ad26, Ad34, Ad35, Ad48, Ad49, Ad50, and/or Pan9/AdC68.

In one preferred embodiment, all seven hexon hypervariable regions (HVRs) and the fiber knob domain of the fiber protein of Ad5 have been replaced with the corresponding hexon HVRs and fiber knob domains, respectively, of an adenovirus serotype selected from Ad11, Ad15, Ad24, Ad26, Ad34, Ad35, Ad48, Ad49, Ad50, and Pan9/AdC68 for the source of HVRs, and an adenovirus serotype having a lower seroprevalence compared to that of Ad5 for the source of fiber knob domain. In another embodiment, all or a portion of one or more of the seven hexon HVR sequences of Ad5 (SEQ ID NOs: 1-7) have been replaced with all or a portion of one or more of the corresponding hexon HVRs of adenovirus serotypes with lower seroprevalence compared to that of Ad5, having amino acid sequence substantially identical to any one of SEQ ID NOs: 9-60, and all or a portion of the fiber knob domain of Ad5 has been replaced with the corresponding fiber knob domain of an adenovirus serotype having lower seroprevalence compared to that of Ad5. In yet another embodiment, all or a portion of one or more of the seven hexon HVR sequences of Ad5 (SEQ ID NOs: 1-7) have been replaced with all or a portion of one or more of the corresponding hexon HVRs of adenovirus serotypes with lower seroprevalence compared to that of Ad5, having amino acid sequence with at least 90%, or more particularly 95% or 99% sequence identity, to any one of SEQ ID NOs: 9-60, and all or a portion of the fiber knob domain of Ad5 has been replaced with the corresponding fiber knob domain of an adenovirus serotype having lower seroprevalence compared to that of Ad5.

In another preferred embodiment, in addition to a chimeric hexon protein, the adenoviral Ad5 vector possesses a chimeric fiber protein, in which the entire Ad5 fiber knob domain sequence (SEQ ID NO: 8) has been replaced with an adenovirus serotype having a lower seroprevalence compared to that of Ad5 (e.g., Ad11, Ad15, Ad24, Ad26, Ad34, Ad35, Ad48, Ad49, Ad50, and Pan9/AdC68). In another embodiment, in addition to a chimeric hexon protein, the adenoviral Ad5 vector possesses a chimeric fiber protein, in which all or a portion of the Ad5 fiber knob domain is replaced with all or a portion of a fiber knob domain of an adenovirus serotype with lower seroprevalence compared to that of Ad5, having amino acid sequence substantially identical to the sequence of SEQ ID NO: 61 or SEQ ID NO: 62. In yet another embodiment, in addition to a chimeric hexon protein, the adenoviral Ad5 vector possesses a chimeric fiber protein, in which all or a portion of the Ad5 fiber knob domain is replaced with all or a portion of a fiber knob domain of an adenovirus serotype with lower seroprevalence compared to that of Ad5, having amino acid sequence with at least 90%, or more particularly 95% or 99% sequence identity, to the sequence of SEQ ID NO: 61 or SEQ ID NO: 62.

In a most preferred embodiment, the recombinant chimeric Ad5 vector of the invention features all or a portion of all seven HVR1 to HVR7 sequences of Ad5 replaced with corresponding HVR1 to HVR7 sequences of Ad48 (SEQ ID NOs: 13, 21, 27, 35, 43, 50, and 57, respectively) and all or a portion of the Ad5 fiber knob domain sequence replaced with a fiber knob domain of Pan9/AdC68 (SEQ ID NO: 61). In another preferred embodiment, the chimeric hexon protein includes the amino acid sequence of SEQ ID NO: 63 and the chimeric fiber protein includes the amino acid sequence of SEQ ID NO: 64. In one embodiment, the recombinant replication-defective chimeric Ad5 vector of the invention is Ad5HVR48(1-7)KC68, also referred to herein as Ad5HVR48KC68.

In a most preferred embodiment, the recombinant Ad5 vector with chimeric hexon and fiber proteins displays an enhanced ability to evade Ad5-specific NAbs. Therefore, the recombinant chimeric Ad5 vector of the invention exhibits decreased immunogenicity relative to wild-type Ad5 in the presence of an immune response directed against the wild-type Ad5.

In particular embodiments, the homologous corresponding amino acid sequences replacing the Ad5 HVRs and fiber knob domains of the present invention are substantially identical (e.g., at least 50%, 60%, 70%, 75%, 80%, 85%, 90%, 95%, 96%, 97%, 98%, 99%, or 100% identical) to the sequence of any one of SEQ ID NOs: 9-60 and SEQ ID NOs: 61-62 for the HVRs and fiber knob domains, respectively. In other particular embodiments, the homologous corresponding amino acid sequences replacing the Ad5 HVRs and fiber knob domains of the present invention have at least 90%, or more particularly 95% or 99% sequence identity, to any one of SEQ ID NOs: 9-60 and SEQ ID NOs: 61-62 for the HVRs and fiber knob domains, respectively.

According to a preferred embodiment, the present invention relates to a recombinant replication-defective Ad5 vector including chimeric hexon and fiber proteins, wherein the recombinant vector further includes a heterologous nucleic acid encoding an antigenic gene product, or fragment thereof. In a most preferred embodiment, upon expression in a host, or in host cells, the antigenic gene product, or fragment thereof, invokes an immune response. In one non-limiting embodiment, the recombinant replication-defective adenovirus of the invention has a capsid including the antigenic gene product or fragment thereof. In another preferred embodiment, the antigenic gene product, or fragment thereof, includes a bacterial, viral, parasitic, or fungal gene product, or fragment thereof.

In an embodiment of all aspects of the invention, the bacterial gene product, or fragment thereof, is from Mycobacterium tuberculosis, Mycobacterium bovis, Mycobacterium africanum, Mycobacterium microti, Mycobacterium leprae, Pseudomonas aeruginosa, Salmonella typhimurium, Escherichia coli, Klebsiella pneumoniae, Bruscella, Burkholderia mallei, Yersinia pestis, or Bacillus anthracia. Examples of preferred gene products, or fragments thereof, from Mycobacterium strains include 10.4, 85A, 85B, 85C, CFP-10, Rv3871, and ESAT-6 gene products or fragments thereof.

In an embodiment of all aspects of the invention, the viral gene product, or fragment thereof, is from a viral family selected from the group consisting of the Flaviviridae family (e.g., a member of the Flavivirus, Pestivirus, and Hepacivirus genera), which includes the hepatitis C virus (HCV), Yellow fever virus; tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus.

In another embodiment of all aspects of the invention, the viral gene product, or fragment thereof, is from the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabiá virus, Tacaribe virus, Tamiami virus, Whitewater Arroyo virus, Chapare virus, and Lujo virus.

In yet other embodiments of all aspects of the invention, the viral gene product, or fragment thereof, is from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus.

In still other embodiments of all aspects of the invention, the viral gene product, or fragment thereof, is from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxviridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or H1N1 swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papillomavirus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively).

In a preferred embodiment of the invention, the viral gene product, or fragment thereof, is from human immunodeficiency virus (HIV), human papillomavirus (HPV), hepatitis C virus (HCV), herpes simplex virus (HSV), cytomegalovirus (CMV), Ebola virus, or Marburg virus. In a most preferred embodiment, the viral gene product, or fragment thereof, from HIV is Gag, Pol, Env, Nef, Tat, Rev, Vif, Vpr, or Vpu.

In another embodiment of all aspects of the invention, the parasitic gene product, or fragment thereof, is from Toxoplasma gondii, Plasmodium falciparum, P. vivax, P. ovale, P. malariae, Trypanosoma spp., or Legionella spp. Examples of particularly preferred parasitic proteins that may be cloned into the vectors of the present invention include those from Plasmodium falciparum, such as the circumsporozoite (CS) protein and Liver Specific Antigens 1 or 3 (LSA-1 or LSA-3).

In still other embodiments of all aspects of the invention, the fungal gene product, or fragment thereof, is from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus. Examples of fungal gene products, or fragments thereof, include any cell wall mannoprotein (e.g., Afmp1 of Aspergillus fumigatus) or surface-expressed glycoprotein (e.g., SOWgp of Coccidioides immitis).

In another aspect, the invention features a method of treating a subject (a vertebrate, e.g., a human) having a disease caused by an infective agent, the method including the administration of recombinant replication-defective chimeric Ad5 vector of the invention to the subject. In a preferred embodiment, the chimeric Ad5 vector of the invention includes a heterologous antigenic gene product, or fragment thereof, which stimulates an immune response against the infective agent in the subject. In one non-limiting example, the administration of the chimeric Ad5 vector of the invention expressing an HIV Gag protein, or fragment thereof, to an HIV-positive subject can stimulate an immune response in the subject against HIV, thereby treating the subject.

In another preferred embodiment, the infective agent causing the disease of the subject to be treated is a bacterium, a virus, a parasite, or a fungus. Most preferably, the infective agent is one of the herein mentioned bacteria, viruses, parasites, or fungi. Non-limiting examples of diseases of the subject to be treated include any human health disease such as tuberculosis, AIDS, cancer, hepatitis, herpes, malaria, hemorrhagic fever, chicken pox, mononucleosis, rabies, measles, mumps, rubella, smallpox, flu, or tetanus. The recombinant replication-defective Ad5 of the invention can be administered to the subject intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, topically, locally, by inhalation, injection, infusion, continuous infusion, localized perfusion bathing target cells directly, catheter, lavage, in creams, or lipid compositions. In one preferred embodiment, the chimeric Ad5 vector is administered intramuscularly to the subject. The subject can be administered at least about 1×10³ viral particles (vp)/dose or between 1×10¹ and 1×10¹⁴ vp/dose, preferably between 1×10³ and 1×10¹² vp/dose, and more preferably between 1×10⁵ and 1×10¹¹ vp/dose.

In yet another embodiment of all aspects of the invention, the vector (e.g., the chimeric Ad5 vector) is administered with a pharmaceutically acceptable carrier or excipient.

### DEFINITIONS

By “adenovirus” is meant a medium-sized (90-100 nm), nonenveloped icosahedral virus that includes a capsid and a double-stranded linear DNA genome. The adenovirus can be a naturally occurring adenovirus (e.g., wild-type Ad5) or a recombinant adenovirus (e.g., the chimeric Ad5 of the invention). The recombinant adenovirus can be immunogenic or non-immunogenic.

By “amount sufficient” is meant an amount of an agent of the invention capable of effecting beneficial or desired results, such as clinical results. For example, an amount of an agent of the invention that includes an antigenic gene product (e.g., the HIV Gag gene product, or fragment thereof) that is sufficient to treat a disease caused by an infective agent is an amount that achieves an immune response directed against the antigenic gene product in a host administered the agent, as compared to the response obtained without administration of the composition or administration of the composition without the antigenic gene product.

By “antigen” is meant a substance or molecule, such as a protein, or fragment thereof, that is capable of inducing an immune response. Preferably, the antigen is a gene product, or fragment thereof, from a bacterial, viral, parasitic, or fungal species.

By “antigenic gene product” is meant any peptide which elicits a high level of interferon-γ (IFN-γ) production compared to other tested peptides in the CD8+ T lymphocyte response assays described in the Examples. For example, an antigenic gene product elicits a level of interferon-γ production that is at least 4-fold higher (e.g., 5-fold, 10-fold, 20-fold, 50-fold higher) than the level of interferon-γ production that is elicited using a non-antigenic peptide. The antigenic gene product may be a bacterial, parasitic, fungal, or viral gene product. In a preferred embodiment, the antigenic gene product is a viral gene product. Non-limiting examples of viral antigenic gene products include all or a portion of the HIV Gag, Pol, Env, Nef, Tat, Rev, Vif, Vpr, and Vpu proteins.

By “capsid” is meant a protein shell or coat of a virus which often adopts a helical or icosahedral structure. The capsid of Ad5, for example, adopts an icosahedral structure and consists of three major structural proteins: hexon, penton, and fiber proteins. The capsid encloses the genetic material of the virus.

By “cell-mediated immune response” means the immunological defense provided by lymphocytes, such as the defense provided by sensitized T cell lymphocytes when they directly attack foreign antigens and secrete cytokines (e.g., IFN-γ), which can modulate macrophage and natural killer (NK) cell effector functions and augment T cell expansion and differentiation. The cell-mediated immune response is one of two branches of the adaptive immune response.

A “chimeric” capsid protein is one that includes a sequence of amino acid residues that is not typically found in the protein as isolated from, or identified in, a wild-type virus (e.g., a wild-type adenovirus).

Throughout this specification and claims, the word “comprise,” or variations such as “comprises” or “comprising,” will be understood to imply the inclusion of a stated integer or group of integers but not the exclusion of any other integer or group of integers.

A “decreased immunogenicity” of a chimeric adenovirus relative to a wild-type adenovirus (e.g., Ad5) is meant a decreased recognition of the chimeric adenovirus by a host immune response (i.e., increased evasion of recognition by a mounted humoral immune response, e.g., a neutralizing antibody produced by the host and directed against the wild-type adenovirus). The decreased immunogenicity, as described herebefore, is meant a decreased recognition of the chimeric adenovirus or a portion thereof (e.g., the replaced HVR(s) of the chimeric adenovirus and/or the replaced fiber knob domain of the chimeric adenovirus) by the immune system of the host.

By “fiber knob” or “fiber knob domain” is meant the receptor-binding domain of the fiber protein. The fiber knob domain is encoded by roughly the last 200 amino acids of the fiber protein (Henry et al., J. Virol. 68: 5239-46, 1994; Xia et al., Structure 15: 1259-70, 1994). The fiber knob includes the sequences necessary for fiber trimerization and moieties specific to adenovirus serotypes.

By “fiber protein” is meant an adenoviral capsid protein, which is bound to, and projects from, a penton protein base. The fiber protein assembles into homotrimers, each fiber protein consisting of a tail, a shaft, and a knob domain (Devaux et al., J. Mol. Biol. 215: 567-88, 1990). The tail anchors the fiber to the penton base. The shaft protrudes from the tail and includes repeating 15-amino acid residue motifs, which are believed to form two alternating beta strands and beta bends (Green et al., EMBO J. 2: 1357-65, 1983). The overall length of the fiber shaft region and the number of 15-amino acid residue repeats differ between adenoviral serotypes, generally ranging from 6 to 22 copies of the repeating motif.

By “gene product” is meant to include mRNAs transcribed from a gene as well as polypeptides translated from those mRNAs.

By “heterologous nucleic acid molecule” is meant any exogenous nucleic acid molecule (e.g., a nucleic acid molecule not normally found in wild-type adenovirus) that can be inserted into the adenoviral vector of the invention for transfer into a cell, tissue, or organism, for subsequent expression of a gene product of interest or fragment thereof encoded by the heterologous nucleic acid molecule. In a preferred embodiment, the heterologous nucleic acid molecule, which can be administered to a cell or subject as part of the present invention, can include, but is not limited to, the following: a nucleic acid molecule encoding an antigenic gene product that is of bacterial, parasitic, fungal, or viral origin (e.g., a nucleic acid molecule encoding the HIV Gag, Pol, Env, Nef, Tat, Rev, Vif, Vpr, or Vpu gene product, or fragment thereof).

By “hexon” or “hexon protein” is meant an adenoviral capsid protein, which is the most abundant of all major structural proteins of the adenoviral capsid. The hexon protein provides structure and form to the adenoviral capsid. Hexon proteins assemble into homotrimers, and twelve copies of the trimeric hexon form each of the 20 sides of the icosahedral capsid (Roberts et al., Science, 1986). The hexon protein is usually around 100 kDa in mass and 960 amino acids in length (Rux et al., J. Virol., 2003). Hexon proteins of different adenovirus serotypes are functional homologs. Structurally, alignment of available hexon sequences and crystal structures of hexons show that all hexons adopt a highly conserved core structure (Rux et al., J. Virol., 2003). The greatest variability of sequence between hexon homologs occurs at seven discrete hypervariable regions (HVRs), which vary in length and sequence between adenoviral serotypes (Crawford-Miksza et al., J. Virol. 70: 1836-44, 1996).

“Humoral immune response” means a form of immunity in which antigenic stimulation results in the secretion of antigen-specific antibodies by B lymphocytes. Humoral immune response also refers to the accessory proteins and events that accompany antibody production, including Th2 activation and cytokine production, affinity maturation, and memory cell generation. The humoral immune response is one of two branches of the adaptive immune response.

By “immune response” is meant any response to an antigen or antigenic determinant by the immune system of a vertebrate subject (e.g., a human). Exemplary immune responses include humoral immune responses (e.g., protective immune response, production of antigen-specific antibodies) and cell-mediated immune responses (e.g., lymphocyte proliferation).

By “neutralizing antibody” or “NAb” is meant an antibody which either is purified from, or is present in, serum and which recognizes a specific antigen and inhibits the effect(s) of the antigen in the host (e.g., a human). As used herein, the antibody can be a single antibody or a plurality of antibodies. For example, the neutralizing antibody can inhibit infectivity of (e.g., cell entry), or gene expression directed by, an adenovirus. Neutralizing antibodies can, for example, exert a substantial deleterious effect on infectivity of, or gene expression directed by, an adenovirus, as compared, for instance, to any effect on any other adenoviral property. The response of neutralizing antibodies against a specific adenovirus can be assessed using, for example, a luciferase-based virus neutralization assay described in the Examples.

By “nonnative amino acid sequence” is meant any amino acid that is not found in wild-type capsid proteins of a given serotype of adenovirus (e.g., Ad5), and which preferably is introduced into a capsid protein (e.g., a hexon protein or region thereof (e.g., a HVR) or a fiber protein or region thereof (e.g., a fiber knob domain)) at the level of gene expression (e.g., by production of a nucleic acid sequence that encodes the nonnative amino acid sequence).

A “pharmaceutically acceptable carrier” is meant a carrier which is physiologically acceptable to a treated mammal (e.g., a human) while retaining the therapeutic properties of the compound with which it is administered. One exemplary pharmaceutically acceptable carrier is physiological saline. Other physiologically acceptable carriers and their formulations are known to one skilled in the art and described, for example, in Remington's Pharmaceutical Sciences (18^{th} edition, A. Gennaro, 1990, Mack Publishing Company, Easton, Pa.), incorporated herein by reference.

By “population” is meant all organisms that both belong to a same species and have ancestry from a same or proximal geographical area. Populations can therefore be largely distinguished based on genetic similarities and/or differences. For example, a population of humans from sub-Saharan Africa can be distinguished from a population of humans of European ancestry or a population of humans from the United States using a program such as STRUCTURE™ (available on the internet at pritch.bsd.uchicago.edu/structure.html), which investigates population structure using multi-locus genotype data. Studies of human populations have revealed a high prevalence of pre-existing Ad5-specific NAbs in human populations, with the presence of pre-existing Ad5 immunity varying from one human population to another (e.g., median Ad5-specific NAb titers are greater than 10-fold higher in a sub-Saharan African population compared with a United States population).

By “portion” is meant a part of a whole. A portion may comprise at least 10%, 20%, 30%, 40%, 50%, 60%, 70%, 80%, 90%, or 95% of the entire length of an amino acid or nucleic acid sequence region. For example, a portion may include at least 5, 10, 20, 30, 40, 50, 60, 70, 80, 90, 100, 200, 300, 400, 500, 600, 700, 800, 900, or 1000 consecutive amino acids or nucleotides of a reference protein or nucleic acid molecule, respectively.

By “recognition” of an adenovirus by a host immune system is meant detection of the adenovirus by a neutralizing antibody, for example, by the interaction of the neutralizing antibody with adenovirus epitope(s).

By “recombinant,” with respect to a vector, such as an adenoviral vector, is meant a vector (e.g., a viral genome that has been incorporated into one or more delivery vehicles, e.g., a plasmid, cosmid, etc.) that has been manipulated in vitro, e.g., using recombinant nucleic acid techniques, to introduce changes to the vector (e.g., to include heterologous nucleic acid sequences such as a sequence encoding an antigenic gene product or fragment thereof, e.g., viral Gag, or fragment thereof) in a viral genome (e.g., a replication-deficient Ad5 genome).

By “seroprevalence” is meant the proportion of subjects within a given population who test positive for an antigen by blood serum analysis of antigen-specific neutralizing antibodies. Although seroprevalence of a given antigen can vary from one population to another, the seroprevalence of a given antigen for a given population is a physical characteristic of the given population.

A “subject” or “host” is a vertebrate, such as a mammal, e.g., a human. Mammals include, but are not limited to, farm animals (such as cows), sport animals, pets (such as cats, dogs, and horses), mice, rats, and primates.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids. In some instances, an amino acid sequence is “substantially identical” if it has 1, 2, or 3 substitutions relative to a reference sequence. In other instances, an amino acid sequence is “substantially identical” if it has 10, 20, 30, 40, 50, 60, 70, 80, 90, or 100 substitutions relative to a reference sequence. For nucleic acids, the length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

Other features and advantages of the invention will be apparent from the following Detailed Description, the drawings, and the claims.

### BRIEF DESCRIPTION OF THE DRAWINGS

FIG. 1 shows the generation of chimeric Ad5KC68 and Ad5HVR48KC68. HVRs derived from Ad48 are shown as black bars. Fiber Knob sequences derived from AdC68 are highlighted in black as well. Both the rAd5KC68 and rAd5HVR48KC68 vectors were produced to high titers.

FIG. 2A shows the in vitro relative neutralizing antibody responses to hexon and/or fiber chimeric Ad5 in mice pre-immunized with Ad5. Median log Ad5, Ad5KC68, Ad5HVR48, Ad5HVR48KC68, and Ad48 NAb titers in 72 C57BL/6 mice preimmunized with Ad5 are represented as box-and-whiskers plot representing the full range, 25%-75% interquartile range (box) and medians (bar). **=p<0.0001 and *=p<0.0016.

FIG. 2B shows the in vitro relative neutralizing antibody responses to hexon and/or fiber chimeric Ad5 in humans from South Africa with pre-existing immunity to Ad5. Median log Ad5-specific, Ad5KC68-specific, Ad5HVR48KC68-specific, Ad5HVR48-specific, and Ad48-specific NAb titers NAb titers in 267 South African serum samples are represented as box-and-whiskers plot. **=p<0.0001 and *=p<0.0016.

FIGS. 3A-C show the cellular responses induced by Ad5HVR48, Ad5KC68, and Ad5HVR48KC68 vectors compared to wild-type Ad5 vector in naïve C57BL/6 mice. Naïve C57BL/6 mice were immunized intramuscularly with 10⁹ vp of the vectors Ad5, Ad5KC68, Ad5HVR48, and Ad5HVR48KC68 expressing SIV Gag. FIG. 3A shows AL11-specific tetramer responses at multiple time points. FIG. 3B shows IFN-γ ELISPOT assays using splenocytes from the mice on day 28 post immunization. FIG. 3C shows ICS assays that measure SIV Gag-specific cellular immune responses.

FIGS. 3D-F show the cellular responses induced by Ad5HVR48, Ad5KC68, and Ad5HVR48KC68 vectors compared to wild-type Ad5 vector in Ad5-preimmunized C57BL/6 mice. Ad5-preimmunized C57BL/6 mice were immunized intramuscularly with 10⁹ vp of the chimeric vectors Ad5, Ad5KC68, Ad5HVR48, and Ad5HVR48KC68 expressing SIV Gag. FIG. 3D shows AL11-specific tetramer responses at multiple time points. FIG. 3E IFN-γ ELISPOT assays using splenocytes from the mice on day 28 post immunization. FIG. 3F shows ICS assays that measure SIV Gag-specific cellular immune responses.

FIG. 3G shows the assessment of Gag-specific antibodies at weeks 0 (white bars) and 4 (black bars) by ELISA for naïve C57BL/6 mice.

FIG. 3H shows the assessment of Gag-specific antibodies at weeks 0 (white bars) and 4 (black bars) by ELISA for Ad5-preimmunized C57BL/6 mice.

FIGS. 4A-D show NAb responses in serum from Ad5-seropositive and Ad5-seronegative individuals pre- and post-Ad5 vaccination. FIG. 4A shows NAb log titers in Ad5-seronegative individuals at week 0 (pre-vaccination). FIG. 4B shows NAb log titers in Ad5-seronegative individuals at week 8 (post-vaccination). FIG. 4C shows NAb log titers in Ad5-seropositive individuals at week 0 (pre-vaccination). FIG. 4D shows NAb log titers in Ad5-seropositive individuals at week 8 (post-vaccination).

### DETAILED DESCRIPTION

It was previously described that pre-existing Ad5-specific immunity uses Ad5-specific NAbs that are directed primarily against the hexon protein (Sumida et al., J. Immunol., 2005; Youil et al., Hum. Gene. Ther., 2002), and an Ad5 vector with hexon HVRs exchanged could largely evade pre-existing Ad5-specific NAbs (see, e.g., U.S. Pat. No. 7,741,099, which is incorporated herein by reference in its entirety). However, NAbs directed against other adenoviral capsid components, particularly the fiber protein, have also been described, although the extent to which they are functionally relevant remains unclear (Cheng et al., J. Virol. 84: 630-8, 2010; Gahery-Segard, J. Virol. 72: 2388-97, 1998; Hong et al., J. Virol. 77: 10366-75, 2003; Sumida et al., J. Immunol. 174: 7179-85, 2005). We have discovered that Ad5-specific NAbs following vaccination and natural infection target both hexon and fiber epitopes. Utilizing neutralization assays with capsid chimeric vectors, we observed that NAb responses to hexon appeared dominant and NAb responses against fiber were subdominant in sera from vaccinated mice, vaccinated humans, and naturally exposed humans. Additionally, we have discovered Ad5 vectors having reduced immunogenicity with respect to adenoviral regions. In particular, we have produced chimeric Ad5 vectors having substitutions of specific regions of the adenoviral hexon protein (e.g., the hexon HVRs) and fiber protein (e.g., the fiber knob domain) that evade pre-existing vector immunity more effectively than Ad5 vectors having substitutions in only the HVRs.

### HVR Substitutions

The hexon protein hypervariable regions (HVRs) are seven surface loops. The hexon variability among adenovirus serotypes is concentrated in these seven loops (Crawford-Miksza et al., J. Virol. 70: 1836-44, 1996). The present invention relates to adenoviral vectors in which one or more of the hexon HVRs of Ad5 are replaced with the hexon HVRs of another adenovirus serotype having a lower seroprevalence compared to Ad5. Adenoviruses that have lower seroprevalence compared to Ad5 include, for example, subgroup B (Ad11, Ad34, Ad35, and Ad50) and subgroup D (Ad15, Ad24, Ad26, Ad48, and Ad49) adenoviruses as well as simian adenoviruses (e.g., Pan9, also known as AdC68). It is to be understood that the invention is not limited to the use of the HVRs of the hereinabove mentioned adenoviral serotypes, but may include substitutions of all or a portion of the HVRs (e.g., HVR1-7) from any other adenovirus identified to have lower seroprevalence relative to the wild-type Ad5. The preferred serotypes that are used to provide their hexon protein, or relevant parts thereof, are Ad11, Ad26, Ad35, Ad48, and Pan9/AdC68; Ad48 and Pan9/AdC68 are the most preferred serotypes.

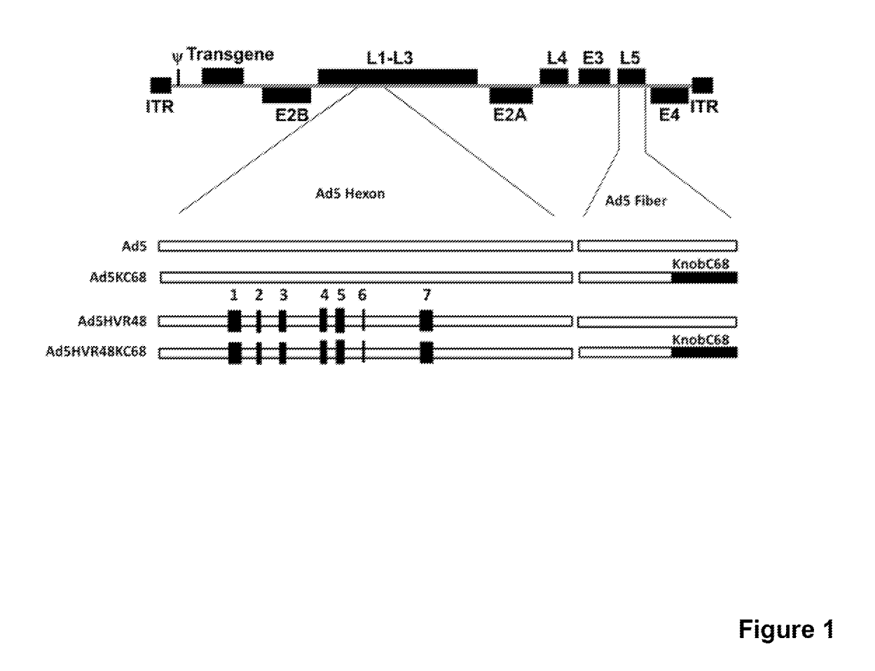

Non-limiting examples of recombinant replication-defective Ad5 vectors of the invention include chimeric Ad5 vectors having at least one, two to five, more preferably six, and most preferably seven HVRs exchanged between serotypes. Preferably at least one HVR from a rare serotype is taken and inserted into the hexon of the backbone wild-type Ad5 serotype. It has been shown previously (U.S. Pat. No. 7,741,099) that replacing all seven HVRs from Ad5 with the corresponding HVRs from Ad48 resulted in a viable and producible vector that encountered lower levels of pre-existing immunity in mice immunized with empty Ad5 viruses relative to wild-type Ad5. However, if only the first HVR (seen from the left ITR to the right ITR in the viral genome, see FIG. 1) was replaced no effect was seen. This does not mean that one replacement could not be enough. It may be that certain individuals raise different immune responses towards different HVRs in comparison to other individuals. Nevertheless, it is most preferred that all HVRs within the Ad5 hexon protein are replaced as this would provide the best chance of yielding a vector not detected by the pre-existing Ad5-specific NAbs present in the host.

In an embodiment, the Ad5 HVR1 sequence, if replaced, is replaced by an amino acid sequence substantially identical to the sequence of any one of SEQ ID NOs: 9-16, or an amino acid sequence with at least 90%, or more particularly 95% or 99% sequence identity, to any one of SEQ ID NOs: 9-16; the Ad5 HVR2 sequence, if replaced, is replaced by an amino acid sequence substantially identical to the sequence of any one of SEQ ID NOs: 17-24, or an amino acid sequence with at least 90%, or more particularly 95% or 99% sequence identity, to any one of SEQ ID NOs: 17-24; the Ad5 HVR3 sequence, if replaced, is replaced by an amino acid sequence substantially identical to the sequence of any one of SEQ ID NOs: 25-30, or an amino acid sequence with at least 90%, or more particularly 95% or 99% sequence identity, to any one of SEQ ID NOs: 25-30; the Ad5 HVR4 sequence, if replaced, is replaced by an amino acid sequence substantially identical to the sequence of any one of SEQ ID NOs: 31-38, or an amino acid sequence with at least 90%, or more particularly 95% or 99% sequence identity, to any one of SEQ ID NOs: 31-38; the Ad5 HVR5 sequence, if replaced, is replaced by an amino acid sequence substantially identical to the sequence of any one of SEQ ID NOs: 39-46, or an amino acid sequence with at least 90%, or more particularly 95% or 99% sequence identity, to any one of SEQ ID NOs: 39-46; the Ad5 HVR6 sequence, if replaced, is replaced by an amino acid sequence substantially identical to the sequence of any one of SEQ ID NOs: 47-52, or an amino acid sequence with at least 90%, or more particularly 95% or 99% sequence identity, to any one of SEQ ID NOs: 47-52; and the Ad5 HVR7 sequence, if replaced, is replaced by an amino acid sequence substantially identical to the sequence of any one of SEQ ID NOs: 53-60, or an amino acid sequence with at least 90%, or more particularly 95% or 99% sequence identity, to any one of SEQ ID NOs: 53-60.

In another embodiment, Ad5 hexon HVRs 1 to 7 are replaced by amino acid sequences from Ad48. These sequences are substantially identical to the sequences of SEQ ID NOs: 13, 21, 27, 35, 43, 50, and 57, respectively, or have amino acid sequences with at least 90%, or more particularly 95% or 99% sequence identity, to any one of the sequences of SEQ ID NOs: 13, 21, 27, 35, 43, 50, and 57, respectively. In yet another embodiment, Ad5 hexon HVRs 1 to 7 are replaced by amino acid sequences from Pan9/AdC68. These sequences are substantially identical to the sequences of SEQ ID NOs: 16, 24, 30, 38, 46, 52, and 60, respectively, or have amino acid sequences with at least 90%, or more particularly 95% or 99% sequence identity, to any one of the sequences of SEQ ID NOs: 16, 24, 30, 38, 46, 52, and 60, respectively.

### Knob Domain Substitutions

The inventors of the present invention discovered that Ad5 NAbs are directed primarily against the hexon HVRs and secondarily against the fiber knob domain in vaccinated mice. The fiber knob domain appeared to account for the vast majority of non-hexon HVR Ad5-specific NAbs. Accordingly, the present invention features recombinant replication-defective Ad5 vectors which, in addition to a chimeric hexon protein, include a chimeric fiber protein in which the fiber knob domain of the fiber protein of Ad5 is substituted with the fiber knob domain of the fiber protein of another adenovirus (e.g., chimpanzee Pan9/AdC68). It is to be understood that the invention is not limited to the use of the fiber knob domain of Pan9/AdC68 as outlined in the Examples. Any fiber knob domain from an adenovirus serotype having a lower seroprevalence compared to Ad5, such as those mentioned hereinabove, can be used to replace the fiber knob domain of Ad5. The preferred serotypes that are used to provide their fiber protein, or relevant parts thereof, are Ad11, Ad15, Ad34, Ad35, Ad48, and Pan9/AdC68; Ad15 and Pan9/AdC68 are the most preferred serotypes.

In an embodiment, the Ad5 fiber knob domain of the Ad5 fiber protein is replaced by an amino acid sequence from Pan9/AdC68 or Ad15. The Pan9/AdC68 or Ad15 sequence is substantially identical to the sequence of SEQ ID NO: 61 or SEQ ID NO: 62, respectively, or an amino acid sequence with at least 90%, or more particularly 95% or 99% sequence identity, to the sequence of SEQ ID NO: 61 or SEQ ID NO: 62, respectively.

Preferred, but not limiting examples of recombinant chimeric replication-defective Ad5 vectors according to the present invention are: Ad5HVR48(1-7)KC68 (also referred to as Ad5HVR48KC68 herein), Ad5HVR48(1-7)K15, Ad5HVRC68(1-7)KC68, Ad5HVRC68(1-7)K15, Ad5HVR48(1-7)K48, Ad5HVRC68(1-7)K48, Ad5HVR11(1-7)KC68, Ad5HVR11(1-7)K15, Ad5HVR11(1-7)K48, Ad5HVR26(1-7)KC68, Ad5HVR26(1-7)K15, Ad5HVR26(1-7)K48, Ad5HVR35(1-7)KC68, Ad5HVR35(1-7)K15, Ad5HVR35(1-7)K48, Ad5HVR48(1-6)KC68, Ad5HVR48(1-6)K15, Ad5HVRC68(1-6)KC68, Ad5HVRC68(1-6)K15, Ad5HVR48(1-6)K48, Ad5HVRC68(1-6)K48, Ad5HVR11(1-6)KC68, Ad5HVR11(1-6)K15, Ad5HVR11(1-6)K48, Ad5HVR26(1-6)KC68, Ad5HVR26(1-6)K15, Ad5HVR26(1-6)K48, Ad5HVR35(1-6)KC68, Ad5HVR35(1-6)K15, and Ad5HVR35(1-6)K48. Ad5HVR48(1-7)KC68 is an exemplary example of a recombinant chimeric replication-defective Ad5 vector of the invention.

Antigens for Insertion into the Chimeric Ad5 Vector

The recombinant replication-defective chimeric Ad5 vectors of the present invention are more efficient for vaccines than vectors solely based on Ad5 or Ad5 vectors with only replaced hexon HVRs. According to a preferred embodiment, the present invention relates to a replication-defective recombinant Ad5 vector including chimeric hexon and fiber proteins, in which the recombinant vector further includes a heterologous nucleic acid encoding an antigenic gene product of interest or fragment thereof. In a preferred embodiment, the antigenic gene product, or fragment thereof, when expressed in a host, or host cells, is capable of eliciting a strong immune response. Non-limiting examples of bacterial gene products, or fragments thereof, include 10.4, 85A, 85B, 86C, CFP-10, Rv3871, and ESAT-6 gene products, or fragments thereof, of Mycobacterium; O, H, and K antigens, or fragments thereof, of E. coli; and protective antigen (PA), or fragments thereof, of Bacillus anthracis. Non-limiting examples of viral gene products, or fragments thereof, include Gag, Pol, Nef, Tat, Rev, Vif, Vpr, or Vpu, or fragments thereof, of HIV and other retroviruses; 9D antigen, or fragments thereof, of HSV; Env, or fragments thereof, of all envelope protein-containing viruses. Non-limiting examples of parasitic gene products, or fragments thereof, include circumsporozoite (CS) protein, gamete surface proteins Pfs230 and Pfs48/45, and Liver Specific Antigens 1 or 3 (LSA-1 or LSA-3), or fragments thereof, of Plasmodium falciparum. Non-limiting examples of fungal gene products, or fragments thereof, include any cell wall mannoprotein (e.g., Afmp1 of Aspergillus fumigatus) or surface-expressed glycoprotein (e.g., SOWgp of Coccidioides immitis).

### Methods of Prophylaxis or Treatment of an Disease Caused By an Infective Agent Using Compositions of the Invention

The compositions of the invention can be used as genetic vaccines for treating a subject with a disease caused by an infective agent such as bacteria, viruses, parasites, and fungi. In particular, the compositions of the invention can be used to treat (pre- or post-exposure) infection by bacteria, including Mycobacterium tuberculosis, Mycobacterium bovis, Mycobacterium africanum, Mycobacterium microti, Mycobacterium leprae, Pseudomonas aeruginosa, Salmonella typhimurium, Escherichia coli, Klebsiella pneumoniae, Bruscella, Burkholderia mallei, Yersinia pestis, or Bacillus anthracis; viruses, in which the virus can be a retrovirus, reovirus, picornavirus, togavirus, orthomyxovirus, paramyxovirus, calicivirus, arenavirus, flavivirus, filovirus, bunyavirus, coronavirus, astrovirus, adenovirus, papillomavirus, parvovirus, herpesvirus, hepadnavirus, poxvirus, or polyomavirus; parasites, including Toxoplasma gondii, Plasmodium falciparum, Plasmodium vivax, Plasmodium ovale, Plasmodium malariae, Trypanosoma spp., or Legionella spp.; or fungi, including Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

In other non-limiting embodiments, the compositions of the invention can be used to treat a subject with tuberculosis, AIDS, cancer, hepatitis, herpes, malaria, hemorrhagic fever, chicken pox, mononucleosis, rabies, measles, mumps, rubella, smallpox, flu, or tetanus.

### Pharmaceutical Formulation and Administration of the Compositions of the Invention

### Administration

The compositions of the invention can be administered to a subject (e.g., a human), pre- or post-exposure to an infective agent (e.g., bacteria, viruses, parasites, fungi), to treat, prevent, ameliorate, inhibit the progression of, or reduce the severity of one or more symptoms of the disease in the subject. Examples of the symptoms of disease, such as a disease caused by viral infection, that can be treated using the compositions of the invention include, e.g., fever, muscle aches, coughing, sneezing, runny nose, sore throat, headache, chills, diarrhea, vomiting, rash, weakness, dizziness, bleeding under the skin, in internal organs, or from body orifices like the mouth, eyes, or ears, shock, nervous system malfunction, delirium, seizures, renal (kidney) failure, personality changes, neck stiffness, dehydration, seizures, lethargy, paralysis of the limbs, confusion, back pain, loss of sensation, impaired bladder and bowel function, and sleepiness that can progress into coma or death. These symptoms, and their resolution during treatment, may be measured by, e.g., a physician during a physical examination or by other tests and methods known in the art.

The compositions utilized in the methods described herein can be formulated for administration by a route selected from, e.g., parenteral, dermal, transdermal, ocular, inhalation, buccal, sublingual, perilingual, nasal, rectal, topical administration, and oral administration. Administration may be, e.g., intramuscular. Parenteral administration includes intravenous, intraperitoneal, subcutaneous, and intramuscular administration. Parenteral, intranasal, or intraocular administration may be provided by using, e.g., aqueous suspensions, isotonic saline solutions, sterile and injectable solutions containing pharmacologically compatible dispersants and/or solubilizers, for example, propylene glycol or polyethylene glycol, lyophilized powder formulations, and gel formulations. The preferred method of administration can vary depending on various factors (e.g., the components of the composition being administered and the severity of the condition being treated). Formulations suitable for oral or nasal administration may consist of liquid solutions, such as an effective amount of the composition dissolved in a diluent (e.g., water, saline, or PEG-400), capsules, sachets, tablets, or gels, each containing a predetermined amount of the chimeric Ad5 vector composition of the invention. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen). In particular, administration by inhalation can be accomplished by using, e.g., an aerosol containing sorbitan trioleate or oleic acid, for example, together with trichlorofluoromethane, dichlorofluoromethane, dichlorotetrafluoroethane, or any other biologically compatible propellant gas.

Immunogenicity of the composition of the invention may be significantly improved if it is co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM matrix, DC-Chol, DDA, cytokines, and other adjuvants and derivatives thereof.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window at the site of release (e.g., the gastro-intestinal tract); or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The compositions of the invention may be administered to provide pre-exposure prophylaxis or after a subject has been exposed to an infective agent, such as a bacterium, virus, parasite, or fungus. The composition may be administered, e.g., 1, 2, 3, 4, 5, 6, 7, 8, 9, 10, 15, 20, 30, 35, 40, 45, 50, 55, or 60 minutes, 2, 4, 6, 10, 15, or 24 hours, 2, 3, 5, or 7 days, 2, 4, 6 or 8 weeks, or even 3, 4, or 6 months pre-exposure, or may be administered to the subject 15-30 minutes or 1, 2, 3, 4, 5, 6, 7, 8, 9, 10, 12, 20, 24, 48, or 72 hours, 2, 3, 5, or 7 days, 2, 4, 6 or 8 weeks, 3, 4, 6, or 9 months, 1, 2, 3, 4, 5, 6, 7, 8, 9, 10, 15, 20 years or longer post-exposure to the infective agent.

When treating disease (e.g., AIDS due to HIV infection, cancer due to HPV infection, malaria due to Plasmodium falciparum infection, etc.), the compositions of the invention may be administered to the subject either before the occurrence of symptoms or a definitive diagnosis or after diagnosis or symptoms become evident. For example, the composition may be administered, e.g., immediately after diagnosis or the clinical recognition of symptoms or 2, 4, 6, 10, 15, or 24 hours, 2, 3, 5, or 7 days, 2, 4, 6 or 8 weeks, or even 3, 4, or 6 months after diagnosis or detection of symptoms.

The compositions may be sterilized by conventional sterilization techniques, or may be sterile filtered. The resulting aqueous solutions may be packaged for use as is, or lyophilized, the lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the recombinant replication-defective chimeric Ad5 vector containing a heterologous nucleic acid encoding an antigenic gene product or fragment thereof (e.g., an Ad5-HIV Gag delivery vector) and, if desired, one or more immunomodulatory agents, such as in a sealed package of tablets or capsules, or in a suitable dry powder inhaler (DPI) capable of administering one or more doses.

### Dosages

The dose of the compositions of the invention (e.g., the number of antigenic gene product-encoding recombinant replication-defective Ad5 delivery vectors) or the number of treatments using the compositions of the invention may be increased or decreased based on the severity of, occurrence of, or progression of, the disease in the subject (e.g., based on the severity of one or more symptoms of, e.g., viral infection).

The pharmaceutical compositions of the invention can be administered in a therapeutically effective amount that provides an immunogenic and/or protective effect against an infective agent or disease caused by an infective agent. For example, the subject can be administered at least about 1×10³ viral particles (vp)/dose or between 1×10¹ and 1×10¹⁴ vp/dose, preferably between 1×10³ and 1×10¹² vp/dose, and more preferably between 1×10⁵ and 1×10¹¹ vp/dose.

Viral particles include nucleic acid molecules encoding an antigenic gene product or fragment thereof (e.g., viral structural and non-structural proteins) and are surrounded by a protective coat (a protein-based capsid with chimeric hexon and fiber proteins). Viral particle number can be measured based on, e.g., lysis of vector particles, followed by measurement of the absorbance at 260 nm (see, e.g., Steel, Curr. Opin. Biotech., 1999).

The dosage administered depends on the subject to be treated (e.g., the age, body weight, capacity of the immune system, and general health of the subject being treated), the form of administration (e.g., as a solid or liquid), the manner of administration (e.g., by injection, inhalation, dry powder propellant), and the cells targeted (e.g., epithelial cells, such as blood vessel epithelial cells, nasal epithelial cells, or pulmonary epithelial cells). The composition is preferably administered in an amount that provides a sufficient level of the antigenic gene product, or fragment thereof, that elicits an immune response without undue adverse physiological effects in the host caused by the treatment.

In addition, single or multiple administrations of the compositions of the present invention may be given (pre- or post-exposure) to a subject (e.g., one administration or administration two or more times). For example, subjects who are particularly susceptible to, e.g., viral infection may require multiple treatments to establish and/or maintain protection against the virus. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against an infective agent, e.g., a bacterium, virus, parasite, or fungus.

Alternatively, the efficacy of treatment can be determined by monitoring the level of the antigenic gene product, or fragment thereof, expressed in a subject (e.g., a human) following administration of the compositions of the invention. For example, the blood or lymph of a subject can be tested for antigenic gene product, or fragment thereof, using, e.g., standard assays known in the art (see, e.g., Human Interferon-Alpha Multi-Species ELISA kit (Product No. 41105) and the Human Interferon-Alpha Serum Sample kit (Product No. 41110) from Pestka Biomedical Laboratories (PBL), Piscataway, N.J.).

A single dose of the compositions of the invention may achieve protection, pre-exposure, from infective agents. In addition, a single dose administered post-exposure to a viral or other infective agent can function as a treatment according to the present invention.

A single dose of the compositions of the invention can also be used to achieve therapy in subjects being treated for a disease. Multiple doses (e.g., 2, 3, 4, 5, or more doses) can also be administered, in necessary, to these subjects.

### Carriers, Excipients, Diluents

The compositions of the invention include a recombinant replication-defective Ad5 vector with chimeric hexon and fiber proteins, containing a heterologous nucleic acid molecule encoding an antigenic gene product or fragment thereof. Therapeutic formulations of the compositions of the invention are prepared using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

### EXAMPLES

The following examples are to illustrate the invention. They are not meant to limit the invention in any way.

### Example 1

### Generation of Recombinant Ad5 Vectors Containing Chimeric Hexon and Fiber Proteins

To evaluate the functional relevance of fiber knob-specific neutralizing antibodies (NAbs) in the suppression of pre-existing Ad5 immunity, we constructed chimeric recombinant Ad5 and Ad5HVR48 vectors in which the fiber knob was exchanged with that of a heterologous virus. Ad5-based vectors with the hexon HVRs and/or the fiber knob exchanged were then evaluated in NAb assays and immunogenicity studies to assess the relative role of hexon- and fiber-specific NAbs following both vaccination and natural infection.

We first constructed chimeric capsid Ad vectors, Ad5KC68 and Ad5HVR48(1-7)KC68 (also referred to as Ad5HVR48KC68 herein) (FIG. 1), in which the Ad5 fiber knob was replaced with that from the chimpanzee adenovirus Pang (AdC68), which has been shown to have low seroprevalence in humans in Africa (Xiang et al., 2006) and also utilize the same primary cellular receptor as Ad5, coxsackievirus adenovirus receptor (CAR) (Cohen et al., J. Gen. Virol. 83: 151-5, 2002), thus ensuring similar receptor usage by all four vectors. Recombinant Ad5 fiber gene fragments were synthesized and cloned into the Ad5 cosmid pWE.Ad5.Aflii-rITR.dE3 or HVR cosmid (pWE.Ad5HVR48.Aflii-rITR.dE3). E1/E3-deleted, replication-incompetent rAd5 vectors containing chimeric hexon and/or fiber knob genes were produced essentially as described (Vogels et al., J. Virol., 2003). The Ad5KC68 and Ad5HVR48KC68 vectors were produced to high titers, and exhibited similar analytical and performance characteristics as compared with Ad5 and Ad5HVR48 vectors in terms of yield, purity, and specific infectivity.

### Example 2

### Determination of NAb Responses to Ad5, Ad5KC68, Ad5HVR48KC68, Ad5HVR48 and Ad48 Viruses in Mice and Humans with Pre-Existing Ad5-Specific Immunity

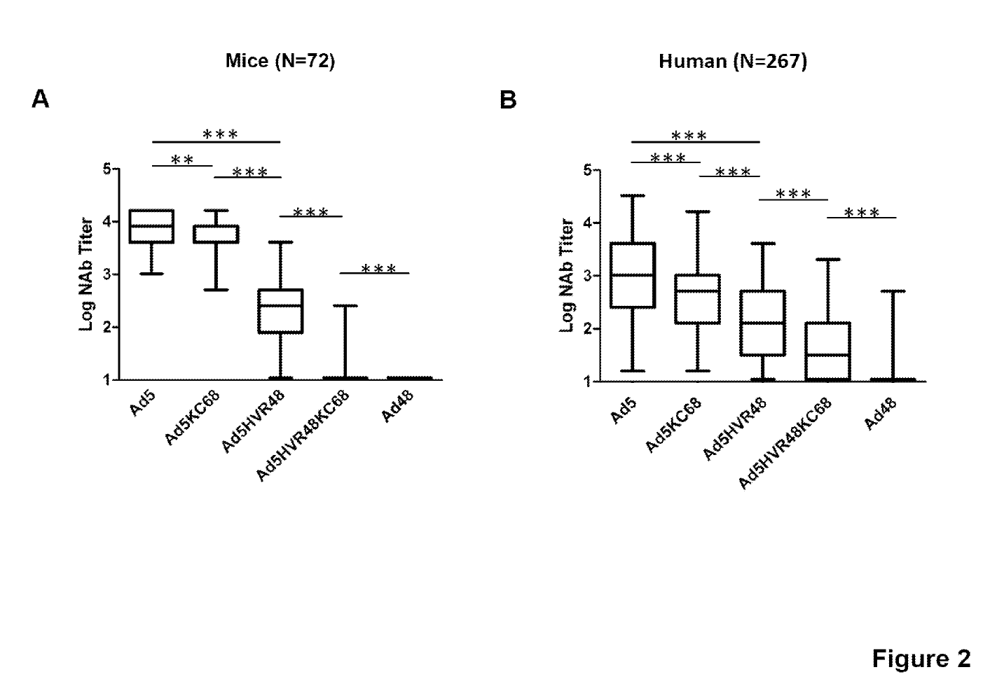

We next evaluated NAb responses against Ad5, Ad5KC68, Ad5HVR48KC68, Ad5HVR48 and Ad48 expressing luciferase using both mouse and human serum samples with a luciferase based virus neutralization assay as described (Vogels et al., J. Virol., 2003). To generate high levels of Ad5-specific immunity, mice were pre-immunized with two injections of 10¹⁰ vp Ad5-Empty separated by 4 weeks. Sera from Ad5 pre-immunized C57BL/6 mice (n=72) were analyzed for NAb titers to these viruses, defined as the serum dilution that neutralized 90% of luciferase activity (FIG. 2A). High Ad5 NAb titers (median log titer 3.9) were detectable in all vaccinated mice, and Ad48 NAb titers were not observed, as expected. Intermediate NAb titers were evident against the chimeric vectors Ad5KC68 and Ad5HVR48. Median Ad5HVR48 NAb titers (median log titer 2.4) were 1.5 log lower than the median Ad5 NAb titer (p<0.0001, Wilcoxon signed-rank test) (FIG. 2A), similar to our previous data (Roberts et al., Nature, 2006). Ad5KC68 NAb titers (median log titer 3.6) proved 0.3 log lower than Ad5 NAb titers (p=0.0016), and 1.2 log higher than Ad5HVR48 NAb median titers (p<0.0001) (FIG. 2A). Ad5HVR48KC68 NAb titers were largely absent. These data indicate that Ad5 NAbs are directed primarily against the hexon HVRs and secondarily against the fiber knob in vaccinated mice. Nevertheless, the fiber knob appeared to account for the vast majority of non-HVR Ad5 NAbs.

We next evaluated NAb responses against Ad5, Ad5KC68, Ad5HVR48KC68, Ad5HVR48 and Ad48 in serum from 267 healthy adults from South Africa (FIG. 2B). Overall, similar results were observed in naturally Ad5-infected humans as compared with vaccinated mice. High Ad5 NAb titers were detected in these samples (median log titer 3.0), indicating high levels of pre-existing Ad5 NAbs as a result of natural Ad5 exposure (Barouch et al., Vaccine 29: 5203-9, 2011). As expected, Ad48 NAb titers were particularly low (Barouch et al., Vaccine 29: 5203-9, 2011). Ad5HVR48 NAb titers (median log titer 2.1) were 1.0 log lower than Ad5 NAb titers (p<0.0001) (FIG. 2B), indicating that approximately 90% of Ad5 NAbs were directed against epitopes located in the seven hexon HVRs. Ad5KC68 NAb titers (median log titer 2.7) were 0.3 log lower than Ad5 titers (p<0.0001) but 0.6 log higher than Ad5HVR48 NAb titers (p<0.0001) (FIG. 2B). Ad5HVR48KC68 Nab titers proved lower than both Ad5KC68 and Ad5HVR48 Nab titers. These data confirm our observations in vaccinated mice and show that Ad5-specific NAbs are directed primarily against HVR epitopes and secondarily against fiber knob epitopes in a large cohort of naturally Ad5-infected humans from Sub-Saharan Africa.

### Example 3

### Determination of Cellular Responses to Ad5, Ad5KC68, Ad5HVR48KC68, Ad5HVR48 and Ad48 Viruses in Naïve and Pre-Immunized Mice

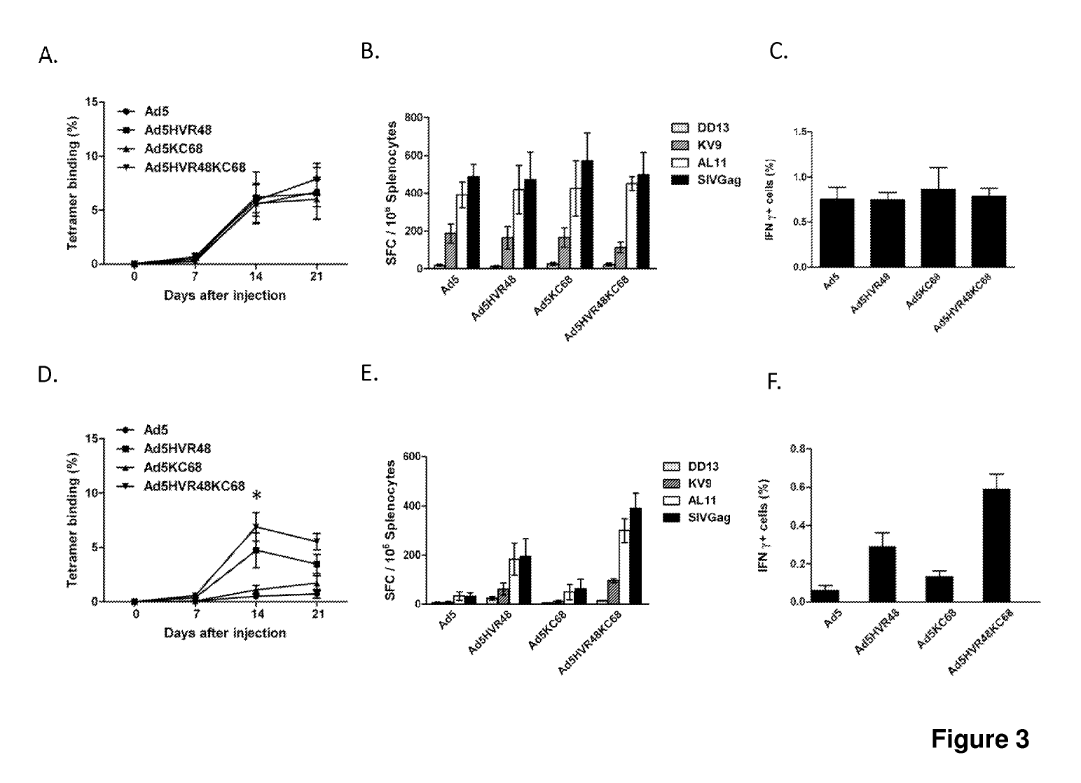

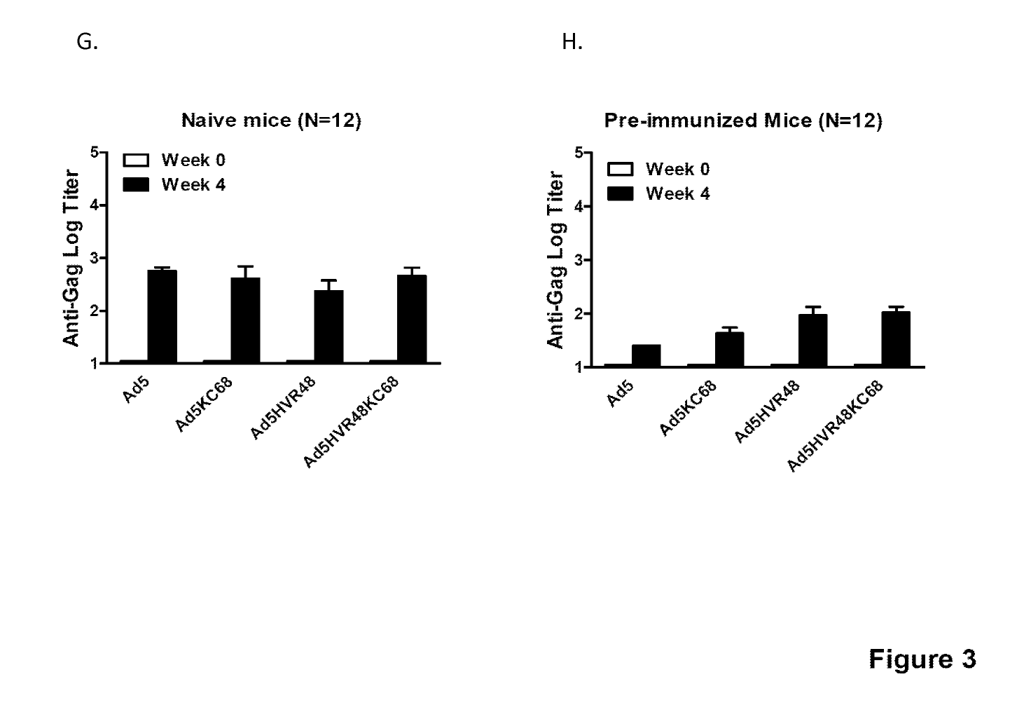

We evaluated the immunogenicity of Ad5, Ad5HVR48, Ad5KC68, and Ad5HVR48KC68 vectors expressing SIV Gag in C57BL/6 mice to evaluate if swapping the fiber knob would improve evasion of anti-Ad5 immunity in vivo. We previously reported that substituting all seven HVRs in Ad5 with those from a rare human adenovirus serotype, Ad48, resulted in a chimeric vector Ad5HVR48(1-7) that evaded the majority of pre-existing Ad5 immunity in preclinical studies in mice and rhesus monkeys (Roberts et al., Nature, 2006). To induce high levels of anti-Ad5 immunity, mice were pre-immunized intramuscularly twice, separated by a 4-week interval, with 10¹⁰ vp of Ad5-Empty in 100 μl sterile PBS (Roberts et al., Nature, 2006). Naive as well as Ad5-pre-immunized C57BL/6 mice (median log Ad5 titer 3.9) (n=8/group) were intramuscularly immunized once with 10⁹ vp of each of these vectors. Tetrameric H-2D^{b} complexes folded around the immunodominant SIV Gag AL11 epitope (AAVKNWMTQTL) (Liu et al., J. Virol. 80: 11991-7, 2006) were prepared and used to measure SIV Gag-specific CD8⁺ T lymphocyte responses on days 0, 7, 14 and 21 post-immunization. CD8⁺ T lymphocytes from naïve mice exhibited <0.1% tetramer staining. In naïve mice, the kinetics and magnitude of AL11-specific CD8⁺ T lymphocyte responses proved comparable with all other vectors (FIG. 3A). To evaluate functional responses, splenocytes from day 28 were utilized in IFN-γ ELISPOT and intracellular cytokine staining (ICS) assays as described (Liu et al., J. Virol. 80: 11991-7, 2006). IFN-γ ELISPOT responses to overlapping Gag peptides, the dominant CD8⁺ T cell epitope AL11 (AAVKNWMTQTL), the sub-dominant CD8⁺ T epitope KV9 (KSLYNTVCV), and the CD4⁺ T cell epitope DD13 (DRFYKSLRAEQTD) (Liu et al., J. Virol. 80: 11991-7, 2006) were comparable among all four vectors (FIG. 3B). IFN-γ ELISPOT ICS responses also proved similar among these vectors (FIG. 3C).

In mice with high baseline Ad5 NAb titers (median titer 3.9), the immunogenicity of Ad5-SIV Gag was abrogated as expected, while the immunogenicity of Ad5HVR48-SIV Gag was largely preserved (FIGS. 3D-3F), consistent with our previous data (Roberts et al., Nature, 2006). The immunogenicity of Ad5KC68-SIV Gag was largely suppressed (FIG. 3D-3F), indicating that evasion of only fiber-specific NAbs was insufficient to circumvent high Ad5 NAb titers. Interestingly, the immunogenicity of Ad5HVR48KC68-SIV Gag was completely preserved and was higher than that of Ad5HVR48-SIV Gag (FIG. 3D-3F). These data suggest that fiber-specific NAbs partially suppress vector immunogenicity and that evasion of fiber knob-specific NAbs is beneficial when dominant NAbs are avoided. We also measured Gag-specific antibody responses in naïve mice as well as Ad5 pre-immunized mice by ELISA (FIGS. 3G and 3H). High titers of Gag-specific antibodies were observed in naïve mice immunized with Ad5-SIV Gag, Ad5KC68-SIV Gag, Ad5HVR48(1-7)-SIV Gag and Ad5HVR48KC68-SIV Gag (FIG. 3G). Sera from mice immunized with Ad5HVR48(1-7)-SIV Gag and Ad5HVR48KC68-SIV Gag had significantly higher titers of Gag-specific antibodies than in mice immunized with Ad5-SIV Gag and Ad5KC68-SIV Gag (FIG. 3H). These data indicate that NAbs against fiber are functionally relevant and that exchanging both the hexon HVRs and the fiber knob allows an improved degree of evasion of preexisting Ad5 immunity. These observations are consistent with the nearly complete evasion of NAb responses to the Ad5HVR48KC68 vector in vitro in both murine and human sera (FIGS. 2A and 2B).

### Example 4

### NAb Responses to Ad5, Ad5KC68, Ad5HVR48KC68, Ad5HVR48 and Ad48 Viruses in Ad5-Seropositive and Ad5-Seronegative Humans Pre-Ad5 and Post-Ad5 Vaccination

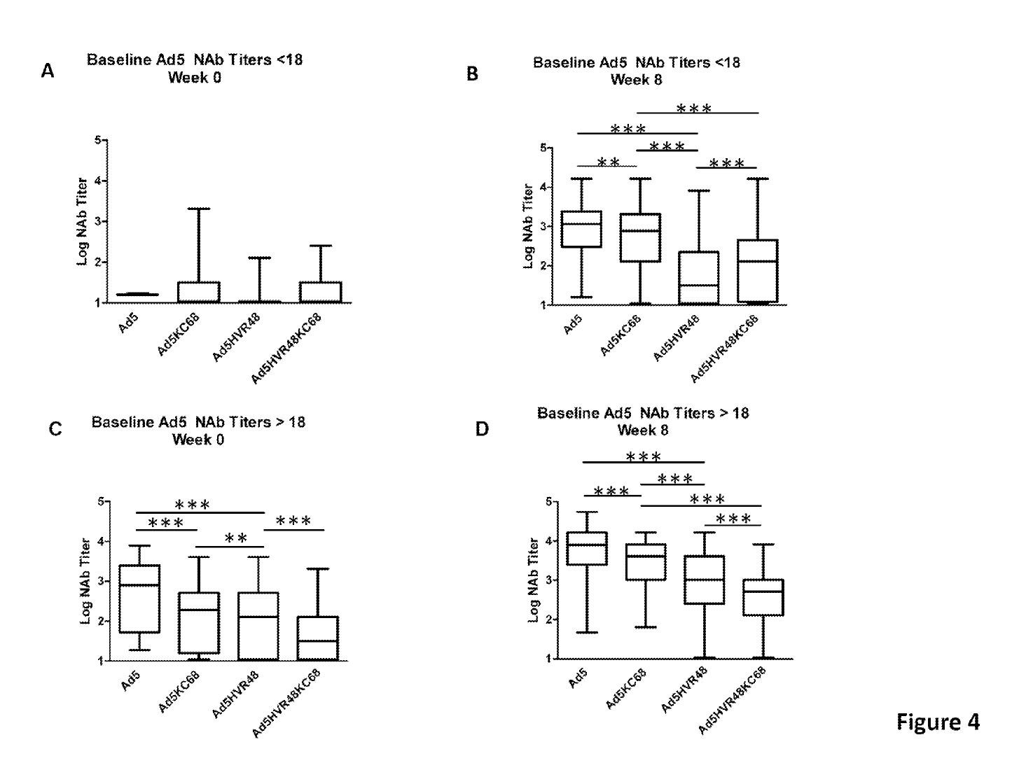

In order to evaluate further the contributions of hexon- and fiber-specific NAbs in the context of both natural Ad5 immunity and vaccine-elicited Ad5 immunity in humans, we analyzed serum samples from 116 subjects vaccinated with the Merck rAd5-Gag vaccine in the phase 1 studies that preceded the STEP study (O'Brien et al., Nat. Med., 2009; Priddy et al., Clin. Infect. Dis., 2008) (FIGS. 4A-4D). We assessed NAb responses against Ad5, Ad5KC68, Ad5HVR48KC68, Ad5HVR48 and Ad48 in serum obtained at week 0 (baseline) and week 8 (4 weeks following the second vaccination). In individuals with baseline Ad5 NAb titers <18, NAbs to all 4 viruses were low to undetectable, as expected, given the relatively low seroprevalence of Ad48 and AdC68 (FIG. 4A). Following vaccination, these individuals exhibited high Ad5-specific NAbs (median log titer 3.0). Ad5 titers were 0.3 log higher than those induced against Ad5KC68 (median log titer of 2.7) (p=0.0016) but were 1.5 log higher than those against Ad5HVR48 (median log titer 1.5) (p<0.0001), and 1.0 log higher than those against Ad5HVR48KC68 (p<0.0001) (FIG. 4B). These data indicate that in Ad5 naïve individuals, the majority of Ad5-specific NAbs induced by vaccination are directed primarily against the hexon, although low levels of fiber-specific and other NAbs were also detected.

In individuals with baseline Ad5 NAb titers >18, high Ad5-specific NAbs were detected (median log titer of 2.9) at week 0 (FIG. 4C). Consistent with the prior experiments, these were 0.4 log higher than those against Ad5KC68 (median log titer of 2.5) (p<0.0001), 0.6 log higher than those against Ad5HVR48, (p<0.0001), and 1.4 log higher than those against Ad5HVRKC68 (p<0.0001) (FIG. 4C). Following vaccination, a similar stepwise hierarchy of NAb titers was observed (FIG. 4D), suggesting that the dominance of HVR-specific NAbs and sub-dominance of fiber knob-specific NAbs were similar among vaccinated mice, naturally infected humans, and vaccinated humans.

These data confirm and extend previous studies showing that Ad5-specific NAbs are directed primarily against the hexon HVRs (Roberts et al., Nature, 2006). However, Ad5-specific NAbs against the fiber knob also exist and appear to be functionally relevant but secondary in nature to hexon-specific NAbs (Barouch et al., J. Immunol. 172: 6290-7, 2004; Cheng et al., J. Virol. 84: 630-8, 2010; Gahery-Segard, J. Virol. 72: 2388-97, 1998; Hong et al., J. Virol. 77: 10366-75, 2003; Sumida et al., J. Immunol. 174: 7179-85, 2005). Specifically, simply replacing the Ad5 fiber knob with that of a heterologous Ad vector was insufficient to evade high levels of Ad5-specific NAbs in mice. However, replacing the fiber knob in addition to the seven hexon HVRs of the hexon resulted in a chimeric vector that evaded pre-existing Ad5 immunity even more effectively than did Ad5HVR48 in mice. These observations are consistent with the dramatic reduction of NAb titers to the Ad5HVR48KC68 vector in both vaccinated mice (FIG. 2A) and naturally infected humans (FIG. 2B).

A previous study reported that Ad5-specific NAbs induced by natural infection were primarily directed against Ad5 fiber, whereas Ad5-specific NAbs elicited by vaccination were primarily directed against non-fiber capsid components (Cheng et al., J. Virol. 84: 630-8, 2010). In contrast to this report, we observed that Ad5-specific NAbs were in fact directed primarily against hexon in both vaccinated and naturally infected individuals in two large cohorts of human subjects (FIGS. 2B and 4C). Our data thus suggest that there is no fundamental differential targeting of capsid-specific NAbs by Ad5 vector vaccination compared with natural infection. The differences between these two studies may relate to different sample sizes, study populations, or chimeric vector characteristics.

Identification of key neutralizing epitopes in the adenovirus capsid will lead to a better understanding of adenovirus immunology as well as improved adenovirus based vectors for vaccination and gene therapy. Our findings indicate that NAbs, whether induced naturally or by a vaccine vector, are directed primarily against the hexon HVRs, while residual NAbs are largely directed against the fiber knob. The near complete evasion of Ad5 NAbs by the Ad5HVR48KC68 vector also warrants further investigation as a novel candidate vector.

### Example 5

### Prophylaxis or Treatment of Tuberculosis Using the Compositions of the Invention

Tuberculosis (TB) is a disease caused by various strains of mycobacteria, usually Mycobacterium tuberculosis. The mode of transmission of TB is often via the air, when people suffering from active TB, for example, cough, sneeze, or spit, thereby transmitting the disease. Humans of both sexes and all ages are susceptible to TB, and infected humans may develop cough, chest pain, weight loss, fatigue, fever, chills, or loss of appetite symptoms. Although a bacillus Calmette-Guerin (BCG) vaccine is available in some countries where TB is more common, the vaccine is most effective in preventing severe TB in infants, but less effective in adults.

The compositions of the invention could be administered to a subject (e.g., a human) with TB or for prophylactic treatment of TB. In this embodiment, the recombinant replication-defective chimeric Ad5 vector includes a heterologous nucleic acid sequence encoding all or a portion of an antigenic M. tuberculosis gene product (e.g., CFP-10, Rv3871, ESAT-6, 10.4, 85A, 85B, or 86C). Upon expression in a subject, the antigenic M. tuberculosis protein, or fragment thereof, will invoke an immune response in the subject. This immune response is required for different kinds of vaccination settings. One example is post-exposure TB treatment, in which the immune response towards the antigenic M. tuberculosis protein of interest adds to the removal of M. tuberculosis which expresses the protein. Another preferred application is in prophylactic treatment such as vaccination to prevent or to significantly inhibit the infection of the subject by M. tuberculosis. Thus, the compositions of the invention (e.g., a recombinant chimeric Ad5 vector containing M. tuberculosis CFP-10 protein) could be administered to the subject to prevent, treat, or reduce one or more of the hereinabove-mentioned symptoms of TB in the subject. These symptoms, and their resolution during treatment, may be measured by, e.g., a physician during a physical examination or by other tests and methods known in the art.

The dose of the compositions of the invention (e.g., the number of M. tuberculosis CFP-10-encoding chimeric Ad5 delivery vectors) or the number of treatments using the compositions of the invention may be increased or decreased based on the severity of, occurrence of, or progression of, the disease or symptoms in the subject.

### Example 6

### Prophylaxis or Treatment of AIDS Using the Compositions of the Invention

The compositions of the invention could be administered to a subject (e.g., a human) with AIDS. In this preferred embodiment, the recombinant replication-defective chimeric Ad5 vector includes a heterologous nucleic acid sequence encoding all or a portion of an antigenic HIV gene product (e.g., Gag, Pol, Nef, Tat, Rev, Vif, Vpr, or Vpu). Upon expression in a subject, the antigenic HIV protein, or fragment thereof, will invoke an immune response in the subject. This immune response is required for different kinds of vaccination settings. One example is post-exposure HIV treatment, in which the immune response towards the antigenic HIV protein of interest adds to the removal of HIV which expresses the protein. Another preferred application is in prophylactic treatment such as vaccination to prevent or to significantly inhibit the infection of the subject by HIV. Thus, the compositions of the invention (e.g., a recombinant chimeric Ad5 vector containing HIV Gag protein) could be administered to the subject to prevent, treat, or reduce one or more symptoms of AIDS in the subject. Examples of the symptoms of AIDS that could be treated or reduced include, e.g., fatigue, weight loss, headaches, chronic diarrhea, blurred or distorted vision, fever, chills, skin rashes or bumps, shortness of breath, and low T cell count in an HIV-positive subject or subject with AIDS. As mentioned hereinabove, these symptoms, and their resolution during treatment, may be measured by, e.g., a physician during a physical examination or by other tests and methods known in the art.

The dose of the compositions of the invention (e.g., the number of HIV Gag-encoding chimeric Ad5 delivery vectors) or the number of treatments using the compositions of the invention may be increased or decreased based on the severity of, occurrence of, or progression of, the disease or symptoms in the subject.

### Example 7

### Prophylaxis or Treatment of Malaria Using the Compositions of the Invention

Plasmodium falciparum is a protozoan parasite which is the primary cause of the most severe and fatal forms of malaria in humans. The primary mode of transmission is via the bite of an infective Anopheles mosquito (e.g., Anopheles gambiae). Humans of both sexes and all ages are susceptible, and when infected with the parasite may develop recurrent attacks of shaking chills, high fever, and/or profuse sweating concomitant with a drop in body temperature as well as headaches, nausea, vomiting, and/or diarrhea. Although a human who live in regions where malaria is common may acquire a partial immunity, the immunity can disappear if the human moves to a region where malaria is less prevalent. Currently, there is no approved vaccine for malaria.

The compositions of the invention could be administered to a subject (e.g., a human) with malaria. In this preferred embodiment, the recombinant replication-defective chimeric Ad5 vector includes a heterologous nucleic acid sequence encoding all or a portion of an antigenic P. falciparum gene product (e.g., circumsporozite (CS) protein, or a fragment thereof). Upon expression in a subject, the antigenic P. falciparum protein, or fragment thereof, will invoke an immune response in the subject. This immune response is required for different kinds of vaccination settings. One example is post-exposure P. falciparum treatment, in which the immune response towards the antigenic P. falciparum protein of interest adds to the removal of P. falciparum which expresses the protein. Another preferred application is in prophylactic treatment such as vaccination to prevent or to significantly inhibit the infection of the subject by P. falciparum. Thus, the compositions of the invention (e.g., a recombinant chimeric Ad5 vector containing P. falciparum CS protein) could be administered to the subject to prevent, treat, or reduce one or more of the hereinabove-mentioned symptoms of malaria in the subject. These symptoms, and their resolution during treatment, may be measured by, e.g., a physician during a physical examination or by other tests and methods known in the art.

The dose of the compositions of the invention (e.g., the number of P. falciparum CS-encoding chimeric Ad5 delivery vectors) or the number of treatments using the compositions of the invention may be increased or decreased based on the severity of, occurrence of, or progression of, the disease or symptoms in the subject.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All patents, patent applications, patent application publications, and other publications cited or referred to in this specification are herein incorporated by reference to the same extent as if each independent patent, patent application, patent application publication, or publication was specifically and individually indicated to be incorporated by reference. Such patent applications specifically include U.S. Provisional Patent Application No. 61/533,029, filed on Sep. 9, 2011, from which this application claims benefit.

1. A recombinant replication-defective adenovirus based upon adenovirus serotype 5 (Ad5), said recombinant replication-defective adenovirus comprising:
(a) a chimeric hexon protein, wherein all or a portion of one or more hexon protein hypervariable region (HVR) sequences of HVR1 to HVR7 of Ad5 have been replaced with all or a portion of one or more of the corresponding hexon HVR sequences from an adenovirus serotype having a lower seroprevalence relative to Ad5, and
(b) a chimeric fiber protein, wherein all or a portion of an Ad5 fiber knob domain sequence has been replaced with all or a portion of a fiber knob domain sequence from an adenovirus serotype having a lower seroprevalence relative to the Ad5.

2. The recombinant replication-defective adenovirus of claim 1, wherein said adenovirus serotype having a lower seroprevalence relative to Ad5 is selected from the group consisting of Ad11, Ad15, Ad24, Ad26, Ad34, Ad35, Ad48, Ad49, Ad50, and Pan9/AdC68.

3. The recombinant replication-defective adenovirus of claim 1, wherein all of the HVR sequences of HVR1 to HVR7 of Ad5 have been replaced with corresponding HVR sequences of an adenovirus serotype selected from the group consisting of Ad11, Ad15, Ad24, Ad26, Ad34, Ad35, Ad48, Ad49, Ad50, and Pan9/AdC68.

4. The recombinant replication-defective adenovirus of claim 1, wherein:
(a) all or a portion of the HVR1 sequence of Ad5 (SEQ ID NO 1) has been replaced by an amino acid sequence substantially identical to the sequence of any one of SEQ ID NOs: 9-16, and/or
(b) all or a portion of the HVR2 sequence of Ad5 (SEQ ID NO: 2) has been replaced by an amino acid sequence substantially identical to the sequence of any one of SEQ ID NOs: 17-24, and/or
(c) all or a portion of the HVR3 sequence of Ad5 (SEQ ID NO: 3) has been replaced by an amino acid sequence substantially identical to the sequence of any one of SEQ ID NOs: 25-30, and/or
(d) all or a portion of the HVR4 sequence of Ad5 (SEQ ID NO 4) has been replaced by an amino acid sequence substantially identical to the sequence of any one of SEQ ID NOs: 31-38, and/or
(e) all or a portion of the HVR5 sequence of Ad5 (SEQ ID NO: 5) has been replaced by an amino acid sequence substantially identical to the sequence of any one of SEQ ID NOs: 39-46, and/or
(f) all or a portion of the HVR6 sequence of Ad5 (SEQ ID NO: 6) has been replaced by an amino acid sequence substantially identical to the sequence of any one of SEQ ID NOs: 47-52, and/or
(g) all or a portion of the HVR7 sequence of Ad5 (SEQ ID NO: 7) has been replaced by an amino acid sequence substantially identical to the sequence of any one of SEQ ID NOs: 53-60.

5. The recombinant replication-defective adenovirus of claim 4, wherein:
(a) all or a portion of the HVR1 sequence of Ad5 (SEQ ID NO: 1) has been replaced by an amino acid sequence having at least 90% sequence identity, or more particularly 95% or 99% sequence identity, to any one of SEQ ID NOs: 9-16, and/or
(b) all or a portion of the HVR2 sequence of Ad5 (SEQ ID NO 2) has been replaced by an amino acid sequence having at least 90% sequence identity, or more particularly 95% or 99% sequence identity, to any one of SEQ ID NOs: 17-24, and/or
(c) all or a portion of the HVR3 sequence of Ad5 (SEQ ID NO: 3) has been replaced by an amino acid sequence having at least 90% sequence identity, or more particularly 95% or 99% sequence identity, to any one of SEQ ID NOs: 25-30, and/or
(d) all or a portion of the HVR4 sequence of Ad5 (SEQ ID NO: 4) has been replaced by an amino acid sequence having at least 90% sequence identity, or more particularly 95% or 99% sequence identity, to any one of SEQ ID NOs: 31-38, and/or
(e) all or a portion of the HVR5 sequence of Ad5 (SEQ ID NO: 5) has been replaced by an amino acid sequence having at least 90% sequence identity, or more particularly 95% or 99% sequence identity, to any one of SEQ ID NOs: 39-46, and/or
(f) all or a portion of the HVR6 sequence of Ad5 (SEQ ID NO 6) has been replaced by an amino acid sequence having at least 90% sequence identity, or more particularly 95% or 99% sequence identity, to any one of SEQ ID NOs: 47-52, and/or
(g) all or a portion of the HVR7 sequence of Ad5 (SEQ ID NO: 7) has been replaced by an amino acid sequence having at least 90% sequence identity, or more particularly 95% or 99% sequence identity, to any one of SEQ ID NOs: 53-60.

6. The recombinant replication-defective adenovirus of claim 1, wherein all of the Ad5 fiber knob domain sequence has been replaced.

7. The recombinant replication-defective adenovirus of claim 1, wherein all or a portion of the Ad5 fiber knob domain sequence (SEQ ID NO: 8) has been replaced with all or a portion of an amino acid sequence substantially identical to the sequence of SEQ ID NO: 61 or SEQ ID NO: 62.

8. The recombinant replication-defective adenovirus of claim 1, wherein all or a portion of the Ad5 fiber knob domain sequence (SEQ ID NO: 8) has been replaced with all or a portion of an amino acid sequence having at least 90% sequence identity, or more particularly 95% or 99% sequence identity, to the sequence of SEQ ID NO: 61 or SEQ ID NO: 62.

9. The recombinant replication-defective adenovirus of claim 1, wherein:
(a) all or a portion of all seven of the HVR sequences of HVR1 to HVR7 of Ad5 have been replaced with corresponding HVR sequences of Ad48 comprising SEQ ID NOs: 13, 21, 27, 35, 43, 50, and 57, respectively; and
(b) all or a portion of the Ad5 fiber knob domain sequence has been replaced with a corresponding fiber knob domain of Pan9/AdC68 comprising SEQ ID NO: 61.

10. The recombinant replication-defective adenovirus of claim 9, wherein:
(a) said chimeric hexon protein comprises an amino acid sequence of SEQ ID NO: 63, and
(b) said chimeric fiber protein comprises an amino acid sequence of SEQ ID NO: 64.

11. The recombinant replication-defective adenovirus of claim 10, wherein said recombinant replication-defective adenovirus is Ad5HVR48(1-7)KC68.

12. The recombinant replication-defective adenovirus of claim 1, wherein said adenovirus exhibits decreased immunogenicity relative to wild-type Ad5 in the presence of a protective immune response directed against said wild-type Ad5.

13. The recombinant replication-defective adenovirus of claim 1, wherein said adenovirus comprises a genome comprising a heterologous nucleic acid encoding an antigenic gene product of interest or fragment thereof, or wherein said recombinant replication-defective adenovirus comprises a capsid comprising a heterologous antigenic gene product of interest or fragment thereof.

14. The recombinant replication-defective adenovirus of claim 13, wherein said antigenic gene product, or fragment thereof, comprises a bacterial, viral, parasitic, or fungal gene product, or fragment thereof.

15. The recombinant replication-defective adenovirus of claim 14, wherein said bacterial gene product, or fragment thereof, is from Mycobacterium tuberculosis, Mycobacterium bovis, Mycobacterium africanum, Mycobacterium microti, Mycobacterium leprae, Pseudomonas aeruginosa, Salmonella typhimurium, Escherichia coli, Klebsiella pneumoniae, Bruscella, Burkholderia mallei, Yersinia pestis, or Bacillus anthracis.

16. The recombinant replication-defective adenovirus of claim 14, wherein said viral gene product, or fragment thereof, is from a viral family selected from the group consisting of Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillamoviridae, Parvoviridae, Astroviridae, Polyomaviridae, Calciviridae, Reoviridae, and Retroviridae.

17. The recombinant replication-defective adenovirus of claim 16, wherein said viral gene product, or fragment thereof, is from human immunodeficiency virus (HIV), human papillomavirus (HPV), hepatitis C virus (HCV), herpes simplex virus (HSV), cytomegalovirus (CMV), Ebola virus, or Marburg virus.

18. The recombinant replication-defective adenovirus of claim 17, wherein said viral gene product, or fragment thereof, from HIV is Gag, Pol, Env, Nef, Tat, Rev, Vif, Vpr, or Vpu.

19. The recombinant replication-defective adenovirus of claim 14, wherein said parasitic gene product, or fragment thereof, is from Toxoplasma gondii, Plasmodium falciparum, Plasmodium vivax, Plasmodium ovale, Plasmodium malariae, Trypanosoma spp., or Legionella spp.

20. The recombinant replication-defective adenovirus of claim 14, wherein said fungal gene product, or fragment thereof, is from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

21. A method of treating a subject having a disease caused by an infective agent comprising administering the recombinant replication-defective adenovirus of claim 1 to said subject.

22.-33. (canceled)
